(19) United States
(12) Patent Application Publication
(10) Pub. No.: US 2021/0139954 A1
(43) Pub. Date: May 13, 2021

(54) NUCLEIC ACID PRETREATMENT KIT, AND BASE SEQUENCE ANALYSIS METHOD

(71) Applicants: SHIMADZU CORPORATION, Kyoto-shi (JP); KAZUSA DNA RESEARCH INSTITUTE, Kisarazu-shi (JP)

(72) Inventors: Masaki KANAI, Kyoto-shi (JP); Hiroyuki JIKUYA, Kyoto-shi (JP); Tetsuo OHASHI, Kyoto-shi (JP); Shin NAKAMURA, Kyoto-shi (JP); Osamu OHARA, Kisarazu-shi (JP)

(73) Assignees: SHIMADZU CORPORATION, Kyoto-shi (JP); KAZUSA DNA RESEARCH INSTITUTE, Kisarazu-shi (JP)

(21) Appl. No.: 17/155,158

(22) Filed: Jan. 22, 2021

Related U.S. Application Data
(62) Division of application No. 16/071,006, filed on Jul. 18, 2018.

(30) Foreign Application Priority Data
Jan. 19, 2016 (JP) 2016-008016

(51) Int. Cl. C12Q 1/6806; C12N 15/09; C12N 15/10; C12M 1/26; C12Q 1/6816; C12Q 1/6834; C12Q 1/6869; C12Q 1/6876

(52) U.S. Cl. CPC C12Q 1/6806; C12N 15/09; C12N 15/1006; C12M 1/26; C12Q 1/6876; C12Q 1/6816; C12Q 1/6834; C12Q 1/6869; C12N 15/1003

(57) ABSTRACT

A pretreatment kit includes a container (10) for particle manipulation, nucleic acid capture particles (70) capable of selectively binding to nucleic acid, aqueous-phase separation medium (21, 22, 23), a plurality of kinds of aqueous liquids (35, 31, 32, 38), and a nucleic acid for individual identification. The nucleic acid for individual identification is contained in at least one of the plurality of kinds of aqueous liquids or is bound onto the surfaces of the nucleic acid capture particles. The base sequence of the nucleic acid for individual identification contains an identification sequence including a base sequence noncomplementary to the nucleic acids contained in the biological sample. The pretreatment kit is used for separating nucleic acids from biological samples that contains nucleic acids and contaminants.

This is a divisional of U.S. application Ser. No. 16/071,006 filed Jul. 18, 2018, which is a National Stage of International Application No. PCT/JP2017/001468 filed Jan. 18, 2017, claiming priority based on Japanese Patent Application No. 2016-008016 filed Jan. 19, 2016.

### TECHNICAL FIELD

The present invention relates to a nucleic acid pretreatment kit for separating and purifying a nucleic acid for a base sequence analysis from a biological sample, and a base sequence analysis method of the nucleic acid separated by using the kit.

### BACKGROUND ART

A base sequence analysis of a nucleic acid contained in a biological sample such as blood, serum, cells, urine, or feces, which is separated and obtained from animals and plants, is useful for medical examination, food safety and hygiene management, or the like. In addition, the relationship between the various diseases and the genomic DNA sequences has been elucidated, and accompanying this elucidation, a base sequence analysis of polymorphism, mutation or the like has been drawing more and more attention.

In performing a base sequence analysis of a nucleic acid in a biological sample, in order to eliminate adverse effects due to a large variety of contaminants such as proteins, sugars, lipids, and the like, it is required to pretreat the biological sample and to separate and purify the nucleic acid. After the separation and purification, the nucleic acid is amplified by a polymerase chain reaction (PCR) method etc., as necessary, and then analyzed by a base sequence analyzer.

A multi-capillary type base sequence analyzer capable of analyzing multiple samples at the same time has been widespread, and it is common to prepare a library including the multiple specimen samples that have been prepared in advance, and to analyze the base sequences of respective samples at the same time. Further, in recent years, next-generation sequencing techniques adopting various principles has been developed, and the ability to analyze a base sequence has dramatically improved.

Since the analysis by a base sequence analyzer is automated, the mixing up of specimen samples due to human error or the contamination between samples are hardly generated at a stage of the base sequence analysis. On the other hand, in a pretreatment for base sequence analysis, such as separation and purification of a nucleic acid from a biological sample, operation such as pipetting, and movement of a sample to a separate container is performed in an open system. Therefore, in the pretreatment operation for a base sequence analysis, contamination between samples may cause, and thus there is a risk of lowering the reliability of the test results.

In Patent Documents 1 to 3, a pretreatment device in which the interior of a container is separated into multiple spaces by an aqueous-phase separation medium, which is insoluble or hardly soluble in water, such as an oil phase or a gel medium, and an aqueous liquid such as a cell lysate, a lavage fluid, or a nucleic acid eluate is loaded into each of the spaces partitioned by the separation medium has been proposed. Specifically, in Patent Document 1, a device in which multiple droplets, and magnetic silica beads that can selectively adsorb a nucleic acid are allowed to be present in an encapsulated medium such as an oil loaded in a container has been proposed. In this device, purification and amplification reaction of nucleic acids can be performed by sequentially moving the magnetic silica beads present in droplets into other droplets by magnetic field manipulation.

In Patent Document 2, a device in which an aqueous liquid layer such as a cell lysate, a lavage fluid, and a nucleic acid eluate, and a gel layer that is hardly soluble or insoluble in water are alternately layered in a tubular container having an open end that can be closed on one end has been proposed. In Patent Document 3, a chip device in which an aqueous liquid layer and a gel layer are alternately arranged in a groove formed on a surface of a substrate has been proposed. In these devices, multiple aqueous liquids are partitioned by a gel, and purification of nucleic acids can be performed by moving magnetic silica beads along the longitudinal direction of a tube sequentially to a cell lysate, a lavage fluid, and a nucleic acid eluate.

In the pretreatment devices disclosed in Patent Documents 1 to 3, the pretreatment of a sample can be performed in a closed system, therefore, the contamination between specimen samples is suppressed, and the reliability of the base sequence analysis can be improved. In addition, these pretreatment devices in a closed system can also be applied to nucleic acid amplification operation such as PCR in addition to separation and purification of nucleic acids from a biological sample. If the separation and purification of nucleic acids from a biological sample and the nucleic acid amplification are performed in the same device in a closed system, the sample obtained by a pretreatment device can be analyzed by a base sequence analyzer as it is, and contamination between samples can be suppressed to the minimum. Further, since multiple kinds of liquids are loaded in one container in a closed system, the number of operations for transferring the sample to another container is few, and the risk of the mixing up of specimen samples due to human error can be reduced.

### PRIOR ART DOCUMENTS

### Patent Documents

Patent Document 1: JP 2008-012490 A

Patent Document 2: WO 2012/086243 A

Patent Document 3: WO 2013/094322 A

### SUMMARY OF THE INVENTION

### Problems to be Solved by the Invention

Even in a case where the pretreatment devices in a closed system disclosed in Patent Documents 1 to 3 are used, a transfer to another container is required at the time of preparing a library for analysis or of injecting a sample into a base sequence analyzer, therefore, there is a risk of the mixing up of specimen samples due to human error. For the purpose of preventing a false detection caused by such a mixing up, a method in which identification information recognizable from the outside by a bar code or the like is attached to each container for loading a specimen sample, and the code management of a process from the preparation of a library up to the base sequence analysis is consistently performed has been widespread.

However, in a method in which the identification information attached to a container is taken over and managed by a process unit, it is difficult to completely eliminate human errors such as mistakes in code reading, and confusion of codes. In the pretreatment device in a closed system as described above, since a large-scale device is not required, it is suitable for use in a clinical site or the like. In contrast, an analysis of a base sequence of a nucleic acid after separation from a biological sample is mostly performed in a place apart from the clinical site. Accordingly, it is difficult to consistently manage a process from the pretreatment of nucleic acids to the analysis of base sequences with a single entity, and a risk of lowering the reliability of a test due to the mistakes in code reading, the confusion of codes, or the like is included.

In view of such problems, an object of the present invention is to provide a method for increasing the reliability of a test by consistently holding the information for specifying a specimen sample in a process from a pretreatment of nucleic acids to a base sequence analysis.

### Means for Solving the Problems

In a pretreatment stage of separating and purifying nucleic acids from a biological sample, a nucleic acid for individual identification having a different base sequence for each specimen is added to a sample, and when analyzing base sequences, in addition to the base sequence of the nucleic acid to be analyzed, the base sequence of the nucleic acid for individual identification is analyzed. In this way, the information for specifying a specimen sample can be consistently held. The present invention relates to a base sequence analysis method applying this method, and a nucleic acid pretreatment kit used for a pretreatment of the base sequence analysis method.

The pretreatment kit is used for separating nucleic acids from a biological sample containing nucleic acids and contaminants. The pretreatment kit includes a container for particle manipulation, nucleic acid capture particles capable of selectively binding to nucleic acids, an aqueous-phase separation medium being insoluble or hardly soluble in water, multiple kinds of aqueous liquids, and a nucleic acid for individual identification.

Two kinds among the multiple kinds of aqueous liquids are a nucleic acid capture liquid for binding nucleic acids contained in a biological sample to nucleic acid capture particles, and a nucleic acid recovery liquid for recovering the nucleic acids bound onto surfaces of the nucleic acid capture particles. The nucleic acid capture liquid may be a cell lysate or the like. The nucleic acid recovery liquid may be a nucleic acid eluate for eluting nucleic acids bound onto surfaces of nucleic acid capture particles from surfaces of the particles, or the like.

Among these constituent elements of the kit, at least the aqueous-phase separation medium and the nucleic acid recovery liquid are contained in a container for particle manipulation. The container for particle manipulation may be configured to be separable in the vicinity of a portion where the nucleic acid recovery liquid is contained.

In the first embodiment of the kit according to the present invention, an aqueous-phase separation medium and all of aqueous liquids are contained in a container for particle manipulation. The second embodiment of the kit according to the present invention includes a container for nucleic acid capture operation in addition to a container for particle manipulation, and the nucleic acid capture liquid is contained in the container for nucleic acid capture operation. The nucleic acid capture particles may be contained in a container, or may be provided separately from the container. In a case where the kit is provided in a state that the nucleic acid capture particles are contained in a container, the nucleic acid capture particles are preferably contained in the nucleic acid capture liquid.

The nucleic acid for individual identification is included in a kit in a state of being contained in at least one of the aqueous liquids, or of being bound onto surfaces of the nucleic acid capture particles. In a case where the nucleic acids for individual identification are contained in a nucleic acid recovery liquid, the recovery rate of the nucleic acids for individual identification is increased. In a case where the nucleic acids for individual identification are contained in the nucleic acid capture liquid or bound onto surfaces of the nucleic acid capture particles, operation is performed in a state that the nucleic acids of a biological sample and the nucleic acids for individual identification coexist from the time of adding the biological sample to the kit, therefore, the reliability of a test is increased. In particular, this is useful in a case where the nucleic acid capture liquid is contained in a container for nucleic acid capture operation, and a sample is moved from the container for nucleic acid capture operation to a container for particle manipulation, as in the second embodiment.

The base sequence of the nucleic acid for individual identification contains an identification sequence including a base sequence noncomplementary to the nucleic acid contained in a biological sample. In base sequence analysis of the nucleic acid in a biological sample recovered by using the kit according to the present invention, the base sequence of the nucleic acid for individual identification is also analyzed. By performing the collation as to whether or not the base sequence of an identification sequence part obtained by a base sequence analysis is consistent with the base sequence of the identification sequence of the nucleic acid for individual identification included in the kit, the reliability of a test is increased.

The nucleic acid for individual identification may contain other sequences on the 3′- and 5′-sides of the identification sequence. In a case where the nucleic acid for individual identification contains a base sequence complementary to the nucleic acid contained in a biological sample on the 3′-side and/or 5′-side of the identification sequence, the nucleic acid of the identification sequence of the nucleic acid for individual identification can also be amplified during the amplification of the nucleic acid contained in a biological sample by PCR, etc.

To the container for particle manipulation, identification information recognizable from the outside of the container is attached. It is preferred that the identification information attached to the container for particle manipulation and the base sequence of the identification sequence are associated with each other. By associating the identification information attached to the container for particle manipulation with the base sequence of the identification sequence, the traceability can be ensured, and further the collation at the time of the base sequence analysis can be easily performed. The identification information is preferably attached in a form recognizable by an optical technique or an electromagnetic technique.

In a case where the nucleic acid capture liquid is contained in a container for nucleic acid capture operation, and the nucleic acids for individual identification are contained in the nucleic acid capture liquid or bound onto surfaces of the nucleic acid capture particles, it is preferred that the identification information is attached to the container for nucleic acid capture operation, and the identification information attached to the container for nucleic acid capture operation and the base sequence of the identification sequence are associated with each other. In addition, it is preferred that the identification information attached to the container for nucleic acid capture operation and the identification information attached to the container for particle manipulation can be associated with each other.

### Effects of the Invention

In the kit according to the present invention, the separation and purification of nucleic acids from a biological sample can be performed in a sealed container, therefore, the contamination between samples can be suppressed even in a case where a large number of specimen samples are handled at the same time. Further, by using the kit according to the present invention, the state that the nucleic acids of a biological sample and the nucleic acids for individual identification coexist is consistently maintained from a pretreatment stage of separating and purifying the nucleic acids from the biological sample to a base sequence analysis.

Even in a case where contamination or mixing up of specimens is generated by any chance, the mixing up of containers and the contamination can be detected by collating the base sequence of an identification sequence part of the nucleic acid for individual identification. In other words, by using the kit according to the present invention, the risk of the contamination or the mixing up of specimens can be reduced, and further the generation thereof can be detected. Therefore, the reliability of a genetic test or the like by a base sequence analysis of nucleic acids is increased.

### BRIEF DESCRIPTION OF THE DRAWINGS

FIG. 1 is a schematic cross-sectional view showing a configuration example of a pretreatment kit of a first embodiment.

FIG. 2 is a schematic cross-sectional view showing a configuration example of a pretreatment kit of a second embodiment.

### MODE FOR CARRYING OUT THE INVENTION

The nucleic acid pretreatment kit according to the present invention is used for a pretreatment of nucleic acids for a base sequence analysis. Specifically, the kit according to the present invention is used for the separation of nucleic acids from a biological sample containing nucleic acids and contaminants. After the separation, the nucleic acids are amplified as necessary, and then subjected to a base sequence analysis.

Examples of the nucleic acid contained in a biological sample include deoxyribonucleic acid (DNA) and ribonucleic acid (RNA). The nucleic acid for individual identification is DNA, RNA, or peptide nucleic acid (PNA). The nucleic acid may be a single-stranded nucleic acid or a double-stranded nucleic acid.

Examples of the biological sample containing nucleic acids include a biological sample such as an animal or plant tissue, a body fluid, and excrement, and a nucleic acid inclusion body such as cells, protozoa, fungi, bacteria, and a virus. The body fluid includes blood, a spinal fluid, saliva, milk, and the like, and the excrement include feces, urine, sweat, and the like. Further, a combination of two or more thereof may also be used. The cells include white blood cells and platelets in blood, exfoliated cells of mucosal cells such as buccal cells, white blood cells in saliva, and the like, and a combination thereof may also be used. The biological sample containing nucleic acids may be prepared in a form of a cell suspension, a homogenate, or a liquid mixture with a cell lysate. These biological samples contain a large variety of contaminants in addition to a nucleic acid being a target substance. For example, in blood, contaminants such as proteins, sugars, lipids, and the like are contained in addition to a nucleic acid.

The pretreatment kit includes nucleic acid capture particles, an aqueous-phase separation medium, an aqueous liquid, and a container for particle manipulation, which accommodates these constituent elements, and further includes a nucleic acid for individual identification.

[Nucleic Acid Capture Particles]

The nucleic acid capture particles are particles capable of selectively binding to nucleic acids. As the particles capable of selectively binding to nucleic acids, silica particles, or particles whose surfaces have been silica coated are preferably used. When nucleic acid capture particles are allowed to be present in a liquid containing nucleic acids and contaminants, the nucleic acids selectively bind to surfaces of the particles. The particles to which nucleic acids have been bound are moved into a lavage fluid, contaminants adhered onto surfaces of the particles are removed, then the particles are moved into a nucleic acid recovery liquid, and the nucleic acids can be recovered in the nucleic acid recovery liquid.

From the viewpoint of facilitating the particle manipulation in a liquid or in a gel medium, the particle diameter of the nucleic acid capture particles is preferably 1 mm or less, and more preferably 0.1 to 500 μm. Although the shape of the particles is desirably a spherical shape with uniform particle diameters, the particles may have irregular shapes and some degree of particle diameter distribution as long as the particle manipulation can be performed.

By the action of a magnetic field, an electric field, a gravitational field, an ultrasonic field or the like, the operation for aggregation, dispersion, movement, and the like of particles can be performed. Among them, the magnetic field manipulation is preferred because aggregation, dispersion, and movement of particles can be easily and accurately performed. In a case where the particle manipulation is performed by a magnetic field, magnetic particles are preferably used as the nucleic acid capture particles. Examples of the magnetic material constituting the magnetic particles include iron, cobalt, and nickel, and a compound thereof, an oxide thereof, and an alloy thereof.

As the nucleic acid capture particles, commercially available magnetic particles may be used. As a commercially available product of the nucleic acid capture magnetic particles whose surfaces are silica coated, Dynabeads (registered trademark) commercially available from Life Technologies Corporation, or MagExtractor (registered trademark) commercially available from TOYOBO CO., LTD can be mentioned.

[Aqueous Liquid]

The aqueous liquid provides a field for chemical operation for immobilization of nucleic acids onto surfaces of nucleic acid capture particles, separation of nucleic acids, and the like. In the separation and purification of nucleic acids in a biological sample by the particle manipulation, a nucleic acid capture liquid for binding the nucleic acids contained in the biological sample to the nucleic acid capture particles, and a nucleic acid recovery liquid for recovering the nucleic acids bound onto surfaces of the nucleic acid capture particles are used. As the aqueous liquid, in addition to the nucleic acid capture liquid and the nucleic acid recovery liquid, a lavage fluid is preferably used. By exposing the particles, onto the surfaces of which nucleic acids have been bound, to a lavage fluid, contaminants and the like that have been adhered onto the surfaces of the particles are removed, and the purity of the nucleic acid can be improved.

<Nucleic Acid Capture Liquid>

The nucleic acid capture liquid provides a field for binding the nucleic acids released into the liquid onto surfaces of nucleic acid capture particles. The nucleic acid capture liquid is preferably one that has a function of breaking the cells and releasing the nucleic acids into the liquid, that is, a cell lysate. As the cell lysate usable as a nucleic acid capture liquid, a buffer solution containing a chaotropic substance, a chelating agent such as ethylenediaminetetraacetic acid (EDTA), tris hydrochloric acid, and the like can be mentioned. In the cell lysate, a surfactant such as Triton X-100 can also be contained. Examples of the chaotropic substance include guanidine hydrochloride, guanidine isothiocyanate, potassium iodide, and urea. In addition to the above, the cell lysate may contain a proteolytic enzyme such as protease K, various kinds of buffer agents, salts, other various kinds of auxiliary agents, an organic solvent such as alcohol, and the like. In the presence of these substances, nucleic acids specifically bind onto surfaces of silica particles or the like. Therefore, if a biological sample containing nucleic acids, and nucleic acid capture particles are added into a cell lysate, the nucleic acids selectively bind to surfaces of the nucleic acid capture particles.

<Lavage Fluid>

The lavage fluid is used for the removal of the contaminants adhered adhered onto surfaces of the particles. As the lavage fluid, any lavage fluid may be accepted as long as it can release components (proteins, sugars, lipids and the like) other than the nucleic acids, a reagent used for dissolving cells, and the like into the lavage fluid while maintaining the state that the nucleic acids are immobilized on surfaces of particles. Examples of the lavage fluid include a high-salt-concentration aqueous solution of sodium chloride, potassium chloride, ammonium sulfate or the like, and an alcohol aqueous solution of ethanol, isopropanol, or the like.

<Nucleic Acid Recovery Liquid>

The nucleic acid recovery liquid is used for recovering the nucleic acids bound onto surfaces of nucleic acid capture particles into the liquid. As a method for recovering the nucleic acids bound onto surfaces of particles into a liquid, a method in which particles are recovered in a state that nucleic acids are bound onto surfaces of the particles, and a method in which nucleic acids bound onto surfaces of particles are eluted into a liquid and recovered as a solution can be mentioned. In a case where the recovery is performed in a state that nucleic acids are bound onto surfaces of the particles, an aqueous solution having a composition similar to that of the above-described lavage fluid is preferably used as the nucleic acid recovery liquid. In a case where nucleic acids are eluted into a liquid, a nucleic acid eluate for eluting nucleic acids bound onto surfaces of nucleic acid capture particles from the surfaces of the particles is used as the nucleic acid recovery liquid.

As the nucleic acid eluate, a buffer solution containing water or a salt at a low concentration can be used. Specifically, a Tris buffer solution, a phosphate buffer solution, distilled water, or the like can be used. Among them, a 5 to 20 mM Tris buffer solution adjusted to have a pH of 7 to 9 is generally used. By moving the particles, onto the surfaces of which nucleic acids have been bound, into a nucleic acid eluate, the nucleic acids are allowed to be eluted from the surfaces of the particles and can be recovered in the nucleic acid eluate.

[Aqueous-Phase Separation Medium]

The aqueous-phase separation medium is arranged in a container so as to be in contact with two or more aqueous liquids, and separates the inside of the container into multiple spaces. An aqueous liquid such as a cell lysate, a lavage fluid, or a nucleic acid recovery liquid is loaded into each of the spaces partitioned by the separation medium.

In order to prevent the contaminations among multiple aqueous media, a substance that is insoluble or hardly soluble in water is used as the aqueous-phase separation medium. The aqueous-phase separation medium may be in a liquid state or a (semi-)solid state, as long as it is insoluble or hardly soluble in water and thus is not mingled with the aqueous liquid.

Examples of the separation medium in a liquid state include hydrocarbons such as an alkane, perfluoroalkanes, a mineral oil, a silicone oil, a fatty acid, a fatty acid ester, a fatty acid amide, a fatty acid ketone, and fatty acid amines. As the separation medium in a (semi-)solid state, one which can be penetrated by nucleic acid capture particles and further with which an aqueous liquid is hardly mixed via through holes of the particles is preferred, and from this viewpoint, a gelled substance is preferable. When particles enter the gel and the particles move in the gel, the gel is perforated, however, the perforations formed in the gel are immediately closed due to a self-repair action by a restoring force of the gel. Accordingly, the inflow of the aqueous liquid via the through holes by the particles is hardly generated.

The material, composition, and the like of the gel that can be used as a separation medium are not particularly limited, and the separation medium may be a physical gel or a chemical gel. For example, as described in WO 2012/086243, a physical gel is formed by heating a liquid substance that is insoluble or hardly soluble in water and adding a gelling agent to the heated liquid substance, and then by cooling the resultant mixture to a sol-gel transition temperature or less. As described in WO 2015/001629, a chemical gel such as a silicone gel has an advantage that the loading into a container is easy, the characteristic changes due to use environment (temperature or the like) are small, and the contaminants derived from the gel are hardly generated.

[Container for Particle Manipulation]

The container for particle manipulation accommodates the aforementioned aqueous liquid and aqueous-phase separation medium. Separation and purification of nucleic acids are performed by the particle manipulation in the container for particle manipulation. The container for particle manipulation has an opening for loading a biological sample or nucleic acid capture particles into the container. The opening of the container for particle manipulation may be capable of closing. By closing the opening and setting the inside of the container as a closed system after the loading of a biological sample and nucleic acid capture particles into the container, contamination from the outside can be prevented. The closing of the opening can be performed by an appropriate method of sealing with a sealing member such as a lid or a plug, thermal fusion of an opening part, or the like.

The material and shape of the container for particle manipulation are not particularly limited as long as the container can hold an aqueous liquid and an aqueous-phase separation medium. Examples of the shape of the container include a tubular shape having an inner diameter of around 1 to 2 mm and a length of around 50 mm to 200 mm, and a shape of a structure in which a flat plate material is bonded to an upper surface of another flat plate material on which a linear groove having a width of around 1 to 2 mm, a depth of around 0.5 to 1 mm, and a length of around 50 mm to 200 mm has been formed.

In a case where the container has a tubular shape, the cross-sectional shape of the tube is not particularly limited, and may be an appropriate shape such as a circular shape, an elliptical shape, a polygonal shape, or the like. As shown in FIG. 1, from the viewpoint of the operability at the time of loading a biological sample and particles into a tubular body, the tubular container may be formed such that the open end has a larger inside diameter. The shape of the container for particle manipulation is not limited to the tubular shape or the planar shape. The container may include a structure in which the movement path of particles has a branch of cross, T shape or the like. Further, a cone-shaped container such as an Eppendorf tube may be used.

Examples of the material for a container for particle manipulation include polyolefin such as polypropylene, and polyethylene; a fluorine-based resin such as tetrafluoroethylene; an organic material such as polyvinyl chloride, polystyrene, polycarbonate, and cyclic polyolefin; and an inorganic material such as ceramic, glass, silicone, and metal. In a case where magnetic particles are used as the nucleic acid capture particles, the particle manipulation is performed by magnetic field manipulation from the outside of a container (for example, movement of a magnet along an outer wall surface of a container). In this case, the material for a container is preferably permeable to the magnetic field. In a case where optical measurement is performed during or after the particle manipulation, or in a case where light irradiation is performed, a material permeable to light is preferably used. In addition, if a container is permeable to light, it is preferred because the state of the particle manipulation inside the container can be visually confirmed.

The container for particle manipulation may be integrally molded or may be configured by a combination of multiple members. In a case where the container is configured by a combination of multiple members, the container may be separable in the combination part. Further, also in a case where the container is integrally molded, the container may be configured to be separable by a method in which a part having a small wall thickness is locally provided to a container (separation line), or the like. In a case where a container for particle manipulation is configured to be separable, by separating a part in which a liquid (nucleic acid recovery liquid) after the recovery of nucleic acids from the other part after the completion of a pretreatment by particle manipulation, the container containing the nucleic acids to be tested is miniaturized, and the storage efficiency can be improved.

[Nucleic Acid for Individual Identification]

The kit according to the present invention includes a nucleic acid for individual identification. The nucleic acid for individual identification contains an identification sequence including a base sequence noncomplementary to the nucleic acids contained in a biological sample. The identification sequences of the nucleic acids for individual identification included in multiple kits, respectively are different from each other. The nucleic acids recovered in a nucleic acid recovery liquid by using a kit include nucleic acids derived from a biological sample and the nucleic acid for individual identification.

In general, a code such as a number for each individual is attached to a kit used for separation and purification of nucleic acids and the like, and a specimen is identified by collating the code attached to the kit with the specimen. The nucleic acid separated by using a kit is transferred from a container for particle manipulation to another container included in the kit, and the amplification and the base sequence analysis are performed. Accordingly, when the mixing up of containers, the contamination, or the like is generated, a result of the analysis of a base sequence of a specimen that is different from the target specimen is brought about.

In a case where the kit according to the present invention is used, the nucleic acids in a nucleic acid recovery liquid includes a nucleic acid derived from a specimen and a nucleic acid for individual identification, and this coexistence state is maintained even when the nucleic acids are transferred to another container in the subsequent processes. In the base sequence analysis, in addition to the base sequence of the nucleic acid (sequence determination target) derived from a specimen, the base sequence of an identification sequence part of the nucleic acid for individual identification is analyzed, and collation is performed as to whether or not the base sequence of an identification sequence part obtained by the analysis is consistent with the base sequence of the identification sequence of the nucleic acid for individual identification contained in the kit. If the base sequence of the identification sequence part is consistent, it can be guaranteed that the target specimen is correctly analyzed.

Although the number of bases of the identification sequence is not particularly limited, the number of bases is preferably 5 or more, and more preferably 7 or more, in order to identify a sufficient number of individuals. In a case of 5 bases, 4⁵=1024 different identification sequences can be generated. It is to be noted, when the identification sequence part specifically hybridizes with a nucleic acid contained in a biological sample, which is a specimen, the subsequent amplification reaction and sequence analysis are adversely affected, and false detection is caused. Accordingly, the identification sequence is selected from the base sequences noncomplementary to the nucleic acid contained in a biological sample.

A nucleic acid for individual identification having a desired base sequence can be synthesized by a known method such as a solid phase synthesis method or a liquid phase synthesis method. In a case where PNA is used as the nucleic acid for individual identification, a nucleic acid for individual identification having a desired sequence may be synthesized using a peptide nucleic acid monomer by a peptide synthesis method such as a 9-fluorenylmethoxycarbonyl (Fmoc) method or a tert-butoxycarbonyl (tBoc) method. The nucleic acid for individual identification may be labeled with a fluorescent label, a radioisotope label, an electrochemical label, an affinity label, an epitope label, or the like.

The nucleic acid for individual identification may contain other base sequences on the 3′-side and/or 5′-side of the identification sequence. For example, in a case where the nucleic acid for individual identification contains a common base sequence on the 3′-side and/or 5′-side of the identification sequence, the common base sequence can be recognized as the start point or end point of the identification sequence. In addition, by including an adapter sequence capable of binding to a flow cell for a base sequence analysis, or a carrier of particles or the like on the 5′-side of the identification sequence, the base sequence of the identification sequence can be easily analyzed.

A base sequence complementary to the nucleic acid (target) to be subjected to a base sequence analysis contained in a biological sample may be included in either one of the 3′- and 5′-sides of the identification sequence. In this case, PCR can be performed using the nucleic acid for individual identification as either a forward (FW) primer or a reverse (RW) primer. Accordingly, the complementary sequence part of the nucleic acid for individual identification anneals with the nucleic acid to be subjected to a base sequence analysis, and a fragment in which the nucleic acid to be analyzed and an antisense sequence of the identification sequence are linked to each other can be amplified. By analyzing the base sequence of this fragment, the base sequence of the nucleic acid to be subjected to a base sequence analysis and the base sequence of the identification sequence of the nucleic acid for individual identification can be analyzed at the same time.

A base sequence complementary to the nucleic acid to be subjected to a base sequence analysis contained in a biological sample may be included on both of the 3′- and 5′-sides of the identification sequence. For example, by using a nucleic acid for individual identification having the same sense sequence as the FW primer on the 3′-side of the identification sequence and an antisense sequence of the RW primer on the 5′-side of the identification sequence, sense and antisense fragments of the nucleic acid for individual identification can be amplified in parallel with the amplification of the nucleic acid to be subjected to a base sequence analysis by PCR.

In a case where a base sequence (complementary sequence) complementary to the nucleic acid to be subjected to a base sequence analysis contained in a biological sample is included on the 3′-side of the identification sequence and an adapter sequence is included on the 5′-side of the identification sequence, that is, in a case where the nucleic acid for individual identification includes an adapter sequence, an identification sequence, and a complementary sequence from on the 5′-side, a fragment having a base sequence of the nucleic acid to be subjected to a base sequence analysis, a base sequence of the identification sequence of the nucleic acid for individual identification, and an adapter sequence is amplified by PCR, and can be analyzed at the same time with the base sequence of the identification sequence of the nucleic acid for individual identification.

As described above, the base sequences other than the identification sequence contained in the nucleic acid for individual identification may be appropriately designed according to a method for analyzing a base sequence, or the like.

### Configuration of Kit and Pretreatment Operation

### First Embodiment

In the first embodiment of the nucleic acid pretreatment kit according to the present invention, an aqueous-phase separation medium and all of aqueous liquids, which constitute the kit, are contained in a container for particle manipulation. In the pretreatment operation using this kit, a biological sample such as blood is added into a container for particle manipulation, and nucleic acids are bound to the nucleic acid capture particles in the container for particle manipulation.

FIG. 1 is a schematic cross-sectional view showing a configuration example of a pretreatment kit of the first embodiment. The pretreatment kit 1 includes a tubular container 10 for particle manipulation, and the container 10 has an opening in the upper part. A nucleic acid recovery liquid 38, a third aqueous-phase separation medium 23, a second lavage fluid 32, a second aqueous-phase separation medium 22, a first lavage fluid 31, a first aqueous-phase separation medium 21, and a nucleic acid capture liquid 35 are loaded from the bottom of the container 10. The aqueous-phase separation media 21, 22, and 23 are gel layers, and the aqueous liquids 31, 32, and 33 are loaded in the spaces partitioned by the inner wall surface of the container and the gel layers.

A lid 13 is attached to an opening part of the container 10 so as to be openable and closable. A biological sample is added into the container 10 in a state that the lid 13 is opened, and then the opening of the container is closed with the lid so that the interior of the container is set to be a closed system.

In the embodiment shown in FIG. 1, magnetic particles 70 as the nucleic acid capture particles are contained in a cell lysate 35 as the nucleic acid capture liquid. The nucleic acid capture particles may be contained in the cell lysate in advance, or the nucleic acid capture particles may be added into the cell lysate immediately before the addition of a biological sample to the cell lysate. In addition, the nucleic acid capture particles may be added into the cell lysate at the same time as or after the addition of a biological sample to the cell lysate. That is, the nucleic acid capture particles may be provided in a state of being included in a kit 1 in advance, or may be provided separately from the kit 1 as one constituent element of the pretreatment kit.

By stirring the cell lysate in a state of containing a biological sample and nucleic acid capture particles and by dispersing the nucleic acid capture particles in the cell lysate, the nucleic acids in the biological sample are bound onto surfaces of the nucleic acid capture particles. The stirring method is not particularly limited, and examples of the method include a method in which a container 10 is vibrated by a vortex mixer or the like, a method in which liquid flow is generated by pipetting or the like, and a method in which particles are moved and stirred in a cell lysate. In a case where the nucleic acid capture particles are magnetic particles, by changing the strength or direction of the magnetic field to be applied from the outside of a container, the particles can be moved and dispersed in a cell lysate.

After binding the nucleic acids onto surfaces of the particles, the particles 70 are moved in the longitudinal direction of a container 10 by magnetic field manipulation. The particles 70 are passed through an aqueous-phase separation medium 21 and moved into a first lavage fluid 31. Washing is performed by dispersing the particles in the lavage fluid. The particles 70 are then passed through an aqueous-phase separation medium 22 and washed in a second lavage fluid 32, and then passed through an aqueous-phase separation medium 23, and moved into a nucleic acid recovery liquid 38. In a case where the nucleic acid recovery liquid 38 is a nucleic acid eluate, by dispersing the nucleic acid capture particles 70 in the nucleic acid eluate, the nucleic acids bound onto surfaces of the particles are eluted, and the nucleic acids can be recovered in the nucleic acid recovery liquid 38.

By including the nucleic acid for individual identification in the nucleic acid recovery liquid 38 in advance, a mixture of the nucleic acid derived from a biological sample (specimen) and the nucleic acid for individual identification is obtained. The nucleic acid for individual identification may also be included in a part other than the nucleic acid recovery liquid 38 in the kit. For example, if the nucleic acids for individual identification are included in a nucleic acid capture liquid (cell lysate), by dispersing the nucleic acid capture particles in the nucleic acid eluate, he nucleic acids for individual identification, as well as the nucleic acids derived from a biological sample, are bound onto surfaces of the particles. Further, if the nucleic acids for individual identification are included in another aqueous liquid such as a lavage fluid, the nucleic acids for individual identification are bound to surfaces of the particles when the nucleic acid capture particles are moved into the aqueous liquid. By moving the nucleic acid capture particles, onto which nucleic acids derived from a biological sample and nucleic acids for individual identification have been bound, into the nucleic acid recovery liquid 38 by magnetic field manipulation or the like, a mixture of the nucleic acids derived from a biological sample and the nucleic acids for individual identification can be recovered in the nucleic acid recovery liquid 38.

Nucleic acids for individual identification may be bound on the surface of the nucleic acid capture particles in advance. The nucleic acid capture particles onto which nucleic acids for individual identification have been bound may be contained in a nucleic acid capture liquid in advance, or the nucleic acid capture particles may also be added into the nucleic acid capture liquid immediately before, at the same time as or after the addition of a biological sample into the nucleic acid capture liquid. In a case where the kit is provided in a state that the nucleic acid capture particles and nucleic acids for individual identification are contained in the nucleic acid capture liquid, the nucleic acids for individual identification are bound onto surfaces of the nucleic acid capture particles.

The nucleic acids recovered in a container for particle manipulation are amplified by a PCR method as necessary, and then analysis of the base sequence is performed by a base sequence analyzer. In a case where an operation after the recovery such as nucleic acid amplification and base sequence analysis is performed in a portion different from a space for the particle manipulation, it is preferred that the nucleic acid sample is moved (delivered) to another portion in a state of being accommodated in the container for particle manipulation from the viewpoint of preventing the contamination or the like caused due to the opening of the container.

In a case where a container for particle manipulation is configured to be separable, the part containing a nucleic acid recovery liquid is separated from the other part, and the container after the separation may be applied for delivery. From the viewpoint of increasing the storage efficiency in a container storage rack etc. used for delivery, it is preferred that the container for particle manipulation is separated in the vicinity of a portion where the nucleic acid recovery liquid is loaded. For example, in the embodiment shown in FIG. 1, it is preferred that a container is separated in a boundary part between a portion where a nucleic acid recovery liquid 38 is loaded and a portion arranged in contact with the nucleic acid recovery liquid 38 where an aqueous-phase separation medium 23 is loaded, or in a portion where an aqueous-phase separation medium 23 is loaded. In a case where the container is separated in a portion where an aqueous-phase separation medium 23 is loaded, the closed system in which the nucleic acid recovery liquid 38 has been enclosed in a space constituted by the inner wall surface of the container after the separation and the aqueous-phase separation medium 23 is maintained, therefore, the risk of contamination can be reduced.

As shown in FIG. 1, it is preferred that identification information 14 recognizable from the outside of a container is attached to the container 10. Examples of the identification information include information that is recognizable by an optical technique with a character string, a bar code, a two-dimensional code or the like, and information that is recognizable by an electromagnetic technique with an integrated circuit (IC) chip, an integrated circuit (IC) tag or the like. This identification information is associated with the base sequence of the nucleic acid for individual identification included in a kit.

The association between the identification information attached to a container and the identification sequence of a nucleic acid for individual identification can be performed by an arbitrary method. In a case where the identification information is a character string, the association may be performed by, for example, a method in which a base sequence of an identification sequence is described as it is as a character string, or a method in which a base sequence is encoded. As an example of encoding a base sequence of DNA, a method in which four kinds of bases (A, G, C, and T) are converted into 2-bit information (00, 01, 10, and 11) to be digitized. Since one base has 2-bit information, identification information of 10 bits can be generated in a case where the identification sequence is 5 bases, and identification information of 20 bits can be generated in a case where the identification sequence is 10 bases. The encoded identification information may be encrypted. Identification information may be attached to a container as numbers or letters, or may be attached to a container as mechanically readable identification information such as a bar code, a two-dimensional code, IC or the like.

The identification information attached to a container and the identification sequence of a nucleic acid for individual identification may be associated with each other via a database. For example, one obtained by encoding a number is attached to a container as identification information, and by making the serial number of the container and the identification sequence of the nucleic acid for individual identification contained in the container into a database, the identification information of the container and the identification sequence can be associated with each other.

The position at which identification information 14 may be attached to a container 10 is not particularly limited, and any position is accepted as long as the identification information can be read after the nucleic acid is recovered in a nucleic acid recovery liquid. In a case where a container for particle manipulation is configured to be separable, it is preferred that the identification information is attached to a position where the unity of the nucleic acid recovery liquid and the identification information can be kept. For example, in a case where the container for particle manipulation is configured to be separable in the vicinity of a portion where the nucleic acid recovery liquid is loaded, it is preferred that the identification information of the container is attached to a portion where the nucleic acid recovery liquid is loaded.

### Second Embodiment

FIG. 2 is a schematic cross-sectional view showing a configuration example of a pretreatment kit of the second embodiment. The pretreatment kit of the second embodiment includes a container 15 for nucleic acid capture operation in addition to a container 10 for particle manipulation. In the above first embodiment, all of the aqueous liquids including the nucleic acid capture liquid and the nucleic acid recovery liquid are contained in a container 10 for particle manipulation. In the second embodiment, in contrast, the nucleic acid capture liquid 35 is contained in a container 15 for nucleic acid capture operation. That is, the pretreatment kit of the second embodiment is configured by a combination of a first kit 101 in which aqueous liquids 31, 32 and 38 and aqueous-phase separation media 21, 22 and 23 are contained in a container 10 for particle manipulation, and a second kit 102 in which a nucleic acid capture liquid 35 is contained in a container 15 for nucleic acid capture operation. In the pretreatment operation using this kit, a biological sample such as blood is added into a container 15 for nucleic acid capture operation, and the binding of nucleic acids to nucleic acid capture particles is performed.

When nucleic acid capture particles such as magnetic silica particles are stored in a liquid for a long period of time, aggregation of particles may cause. Further, the contaminants contained in a biological sample, in particular, a denatured protein produced by the lysis of cells or the like has an effect of masking surfaces of particles, and sticking the particles to each other to aggregate the particles. When the nucleic acid capture particles are aggregated, opportunities of the contact between the surfaces of particles and the nucleic acids are decreased, therefore, the binding of the nucleic acids to the surfaces of particles tends to be inhibited. Accordingly, in order to improve the recovery efficiency of nucleic acids, it is preferred that when nucleic acids are bound to the nucleic acid capture particles, stirring is performed by applying strong vibration to a container so as to crush the aggregate of particles and eliminate the aggregation.

In the kit of the second embodiment, a container 15 for nucleic acid capture operation is prepared separately from the container 10 for particle manipulation in which aqueous-phase separation media 21, 22, and 23, aqueous liquids 31, 32, and 38, and the like have been loaded. The binding of nucleic acids onto surfaces of the nucleic acid capture particles is performed in a state that a nucleic acid capture liquid 35 and magnetic particles 70 are loaded in the container 15 for nucleic acid capture operation. Even if strong vibration is applied to the container 15 for nucleic acid capture operation by using a vortex mixer or the like, aqueous-phase separation media and aqueous liquids, which are loaded in the container 10 for particle manipulation, are not affected at all, and the state that aqueous liquids are loaded in the spaces each partitioned by the inner wall surface of the container and the aqueous-phase separation medium can be maintained. Accordingly, the stirring can be performed by applying an external force stronger than that in a case where the binding of nucleic acids onto surfaces of nucleic acid capture particles is performed in a container for particle manipulation.

Further, the container for nucleic acid capture operation is not required to have a space for accommodating an aqueous-phase separation medium, a nucleic acid recovery liquid, and the like. Therefore, the container may be more compact than the container for particle manipulation, and the degree of freedom of the container shape is high. Accordingly, a shape suitable for dispersing the nucleic acid capture particles into a liquid by stirring can be adopted. Therefore, by performing the binding of nucleic acids onto surfaces of nucleic acid capture particles in a container for nucleic acid capture operation, the dispersion efficiency of the nucleic acid capture particles in a liquid is improved, and the recovery efficiency of nucleic acids can be increased even in a case where the nucleic acid capture particles are aggregated.

The container 15 for nucleic acid capture operation has an opening for adding a biological sample or nucleic acid capture particles into the container and taking out from the container. The opening of the container for nucleic acid capture operation may be capable of closing. An openable and closable sealing member such as a lid or a plug is preferred for closing the opening.

The material and shape of the container for nucleic acid capture operation are not particularly limited as long as the container can hold a nucleic acid capture liquid such as a cell lysate and nucleic acid capture particles. The shape of the container is preferably designed so that particles can be efficiently dispersed in a liquid. As the material for the container, the materials described above as the material for a container for particle manipulation, or the like can be adopted.

In the embodiment shown in FIG. 2, magnetic particles 70 as nucleic acid capture particles are contained in a cell lysate 35 loaded in a container 15 for nucleic acid capture operation. The nucleic acid capture particles may be contained in the cell lysate in advance, or may be added into the cell lysate immediately before the use. Further, the nucleic acid capture particles may be added into a cell lysate at the same time as or after the addition of a biological sample to the cell lysate. In other words, the nucleic acid capture particles may be provided in a state of being included in the second kit 102 in advance, or may be provided separately from the second kit 102 as one constituent element of the pretreatment kit.

The nucleic acids in a biological sample can be bound to the nucleic acid capture particles in a similar manner as in the first embodiment. As described above, in the present embodiment, since the binding of nucleic acids onto surfaces of nucleic acid capture particles is performed in a container for nucleic acid capture operation, the dispersion efficiency of the particles in a liquid can be improved. Accordingly, the amount of nucleic acids bound onto surfaces of the particles can be increased to enhance nucleic acid recovery efficiency.

The nucleic acid capture particles after the binding of nucleic acids are moved into a container 10 for particle manipulation, and subsequently, in a similar manner as in the first embodiment, the particles 70 are moved in the container 10 by magnetic field manipulation, and the nucleic acids are recovered in a nucleic acid recovery liquid 38. When the nucleic acid capture particles are moved from the container 15 for nucleic acid capture operation to the container 10 for particle manipulation, the particles may be moved together with the nucleic acid capture liquid, or only the particles may be moved. In a case where only the particles are moved to the container 10 for particle manipulation, the nucleic acid capture liquid may be adhered onto the surfaces of the particles.

Although an embodiment in which an aqueous-phase separation medium 21 is arranged as the uppermost layer on the open-end side of a container 10 for particle manipulation is shown in FIG. 2, the uppermost layer may be an aqueous liquid such as a lavage fluid. In particular, in a case where only the particles after the binding of nucleic acids are moved from the container for nucleic acid capture operation to the container for particle manipulation, it is preferred that the particles are added into an aqueous liquid arranged on the open-end side of the container, from the viewpoint of simplifying the process of the particle manipulation in the container for particle manipulation.

In the second embodiment as well as in the first embodiment, by including the nucleic acids for individual identification in a kit in advance, a mixture of the nucleic acid derived from a biological sample and the nucleic acid for individual identification can be obtained in the nucleic acid recovery liquid 38. The nucleic acids for individual identification may be contained in any of the container 10 for particle manipulation of the first kit 101 and the container 15 for nucleic acid capture operation of the second kit 102. Further, the nucleic acids for individual identification may be contained in the container 15 for nucleic acid capture operation by adding particles into the container for nucleic acid capture operation, wherein nucleic acids for individual identification have been bound onto the surfaces of the particle in advance. In the second embodiment as well as in the first embodiment, a mixture of the nucleic acid derived from a biological sample and the nucleic acid for individual identification can be recovered into the nucleic acid solution.

The nucleic acids for individual identification may be contained in both of the container for particle manipulation and the container for nucleic acid capture operation. In a case where the nucleic acids for individual identification are contained in both of the container for particle manipulation and the container for nucleic acid capture operation, the identification sequences of the nucleic acids for individual identification may be the same or different. In a case where the identification sequence of the nucleic acid for individual identification contained in the container for particle manipulation and the identification sequence of the nucleic acid for individual identification contained in the container for nucleic acid capture operation are the same, by confirming that only one kind of identification sequence is detected during the base sequence analysis, it can be confirmed that there is no mixing up of specimens and no contamination.

In a case where the identification sequence of the nucleic acid for individual identification contained in the container for particle manipulation and the identification sequence of the nucleic acid for individual identification contained in the container for nucleic acid capture operation are different from each other, by analyzing both of the base sequences of the identification sequences during the base sequence analysis, it can be confirmed that there is no mixing up of specimens and no contamination. The nucleic acid for individual identification contained in the container for particle manipulation and the nucleic acid for individual identification contained in the container for nucleic acid capture operation may have different functions, respectively. For example, if the nucleic acid for individual identification is designed so that one of the nucleic acids for individual identification has a base sequence corresponding to a FW primer in addition to the identification sequence, and the other nucleic acid for individual identification has a base sequence corresponding to a RW primer in addition to the identification sequence, the PCR can be performed by using these two kinds of nucleic acids for individual identification as a pair of primers.

In the second embodiment as well as in the first embodiment, the container 10 for particle manipulation may be configured to be separable in the vicinity of a portion where the nucleic acid recovery liquid is loaded. Further, it is preferred that the identification information 14 associated with the base sequence of the nucleic acid for individual identification is attached to the container 10 for particle manipulation. In a case where the nucleic acids for individual identification are contained in container 10 for particle manipulation, the association between the identification information 14 attached to the container for nucleic acid capture operation and the base sequence of the identification sequence can be performed in a similar manner as in the first embodiment.

In a case where the nucleic acids for individual identification are contained in the container 15 for nucleic acid capture operation (including a case where the nucleic acid capture particles to which nucleic acids for individual identification have been bound are added to the container for nucleic acid capture operation), a mixture of the nucleic acids derived from a biological sample (specimen) and the nucleic acids for individual identification is obtained in the container 15 for nucleic acid capture operation. Accordingly, even if the mixing up of containers is generated when the sample is moved from the container 15 for nucleic acid capture operation to the container 10 for particle manipulation, by analyzing the base sequence of the identification sequence of the nucleic acid for individual identification during the base sequence analysis, the false detection caused by the mixing up can be detected.

In a case where the nucleic acids for individual identification are contained in the container 15 for nucleic acid capture operation, it is preferred that identification information 19 recognizable from the outside of the container is attached to the container 15 for nucleic acid capture operation. It is preferred that the identification information 19 attached to the container 15 for nucleic acid capture operation is associated with the identification sequence of the nucleic acid for individual identification.

In order to ensure the traceability even after the sample in the container 15 for nucleic acid capture operation is moved to the container 10 for particle manipulation, it is preferred that identification informations 14 and 19 are attached to the container 10 for particle manipulation and the container 15 for nucleic acid capture operation, respectively, and these identification informations can be associated with each other. For example, by associating the container 15 for nucleic acid capture operation and the container 10 for particle manipulation with each other in advance, and by attaching the same identification information to the containers, the identification information 19 and the identification information 14 can be associated with each other.

In order to reduce the false detection due to human error such as misreading of identification information or confusion, it is preferred that the identification information 19 of the container 15 for nucleic acid capture operation and the identification information 14 of the container 10 for particle manipulation are associated with each other at the time of use of a kit. For example, when the sample in the container 15 for nucleic acid capture operation is moved to the container 10 for particle manipulation, both of the identification informations can be associated with each other by reading both of the identification informations 19 and 14. Further, the identification information 19 may be peelably attached to the container 15 for nucleic acid capture operation using a seal or the like. In this case, the identification informations 14 and 19 may be associated with each other by peeling the identification information 19 off from the container 15 for nucleic acid capture operation when the sample in the container 15 for nucleic acid capture operation is moved to the container 10 for particle manipulation, and attaching the identification information 19 to the container 10 for particle manipulation.

[Base Sequence Analysis]

In the base sequence analysis method according to the present invention, an analysis of the base sequences of the nucleic acids recovered by the above-described pretreatment kit is performed. The analysis method of the base sequences is not particularly limited. Before the nucleic acids recovered from a pretreatment kit are subjected to a base sequence analysis, a further treatment of fragmentation, ligation, amplification or the like may be performed on the nucleic acids. Such a treatment can be appropriately performed corresponding to the analysis method of base sequences. Even in a case where the nucleic acids recovered from a kit are moved to another container for use in such a treatment, the coexistence state of the nucleic acids derived from a specimen and the nucleic acids for individual identification is maintained.

In the analysis of base sequences, the base sequence of the identification sequence of the nucleic acid for individual identification is analyzed in addition to the base sequence of the nucleic acid in a biological sample. The collation is performed as to whether or not the base sequence of an identification sequence part obtained by the base sequence analysis is consistent with the base sequence of the identification sequence of the nucleic acid for individual identification contained in the kit. If the base sequence of the identification sequence is not consistent, it is considered that the mixing up of specimens or the like is generated. In a case where multiple identification sequences are detected in spite of the fact that only one kind of nucleic acid for individual identification is included in the kit, it is considered that contamination is generated. As described above, by analyzing the base sequence of the identification sequence part, the false detection caused by the mixing up of specimens or the contamination can be detected.

As described above, in the operation for separating and purifying nucleic acids by using the kit according to the present invention, the separation and purification of nucleic acids from a biological sample can be performed in a sealed container, therefore, the risk of trouble due to the contamination between samples, or the like can be reduced even in a case where a large number of specimen samples are handled at the same time. In addition, even in a case where the contamination or the mixing up of specimens is generated by any chance, the false detection based on the contamination, the mixing up, or the like can be detected by collating the base sequence of the identification sequence part of the nucleic acid for individual identification. Accordingly, by performing the separation and purification of nucleic acids by using the kit according to the present invention, the reliability of a genetic test or the like by the base sequence analysis of nucleic acids is increased.

### DESCRIPTION OF REFERENCE SIGNS

10 container for particle manipulation
15 container for nucleic acid capture operation
21, 22, and 23 aqueous-phase separation medium
31 and 32 lavage fluid
35 nucleic acid capture liquid
38 nucleic acid recovery liquid
14 and 19 identification information
70 nucleic acid capture particles

1. A base sequence analysis method using a nucleic acid pretreatment kit for separating a nucleic acid from a biological sample that contains a nucleic acid and a contaminant,
the kit comprising:
a container for particle manipulation;
a nucleic acid capture particle for selectively binding to a nucleic acid;
an aqueous-phase separation medium being insoluble or hardly soluble in water;
a plurality of kinds of aqueous liquids; and
a nucleic acid for individual identification, wherein
two kinds of the plurality of kinds of aqueous liquids are a nucleic acid capture liquid for binding a nucleic acid contained in the biological sample to the nucleic acid capture particle, and a nucleic acid recovery liquid for recovering the nucleic acid attached to a surface of the nucleic acid capture particle,
at least the aqueous-phase separation medium and the nucleic acid recovery liquid are contained in the container for particle manipulation, and
a base sequence of the nucleic acid for individual identification contains an identification sequence including a base sequence noncomplementary to the nucleic acid contained in the biological sample,
the method comprising the following steps:
maintaining a coexistence state of the nucleic acid in the biological sample and the nucleic acid for individual identification in the container for particle manipulation, in a state of being attached to the nucleic acid capture particle;
recovering the nucleic acid in the biological sample and the nucleic acid for individual identification into the nucleic acid recovery liquid; and
analyzing the base sequence of recovered nucleic acid.

2. The base sequence analysis method according to claim 1, further comprising:
analyzing, in an analysis of the base sequence, a base sequence of an identification sequence of the nucleic acid for individual identification as well as the base sequence of the nucleic acid in the biological sample; and
performing a collation as to whether or not the base sequence of an identification sequence part obtained by the base sequence analysis is consistent with the base sequence of the identification sequence of the nucleic acid for individual identification included in the kit.

3. The base sequence analysis method according to claim 1, further comprising:
adding the nucleic acid capture particle into the container for particle manipulation, wherein the nucleic acid for individual identification is attached to the nucleic acid capture particle in advance.

4. The base sequence analysis method according to claim 1, wherein the aqueous-phase separation medium and all of the aqueous liquids are contained in the container for particle manipulation.

5. The base sequence analysis method according to claim 1, the kit further comprising a container for nucleic acid capture operation, wherein the nucleic acid capture liquid is contained in the container for nucleic acid capture operation.

6. The base sequence analysis method according to claim 5, wherein
an identification information recognizable from an outside of a container is attached to the container for particle manipulation, and
the identification information attached to the container for particle manipulation and the base sequence of the identification sequence are associated with each other.

7. The base sequence analysis method according to claim 5, wherein an identification information recognizable from an outside of a container is attached to the container for nucleic acid capture operation, and
the identification information attached to the container for nucleic acid capture operation and the base sequence of the identification sequence are associated with each other.

8. The base sequence analysis method according to claim 7, wherein
an identification information recognizable from an outside of a container is attached to the container for particle manipulation, and
the identification information attached to the container for nucleic acid capture operation and the identification information attached to the container for particle manipulation are capable of being associated with each other.

9. The base sequence analysis method according to claim 6, wherein the identification information of the container for particle manipulation is attached to a portion where the nucleic acid recovery liquid is contained.

10. The base sequence analysis method according to claim 6, wherein the identification information is attached in a form recognizable by an optical technique or an electromagnetic technique.

11. The base sequence analysis method according to claim 1, wherein the aqueous-phase separation medium is a gel.

12. The base sequence analysis method according to claim 1, wherein the nucleic acid capture particle is magnetic particle.

13. The base sequence analysis method according to claim 1, wherein the nucleic acid recovery liquid is a nucleic acid eluate for eluting a nucleic acid attached to a surface of the nucleic acid capture particle from the surface of the particle.

14. The base sequence analysis method according to claim 1, wherein the container for particle manipulation is configured to be separable in a vicinity of a portion where the nucleic acid recovery liquid is contained.

15. The base sequence analysis method according to claim 1, wherein the nucleic acid for individual identification contains a base sequence complementary to a nucleic acid contained in the biological sample on at least one of 3′-side and 5′-side of the identification sequence.
